(19) United States
(12) Patent Application Publication
(10) Pub. No.: US 2015/0190273 A1
(43) Pub. Date: Jul. 9, 2015

(54) Medical Device to Control Environmental Conditions for Surgical and Non-Surgical Wounds

(71) Applicant: Louis Causley Causley, San Jose, CA (US)

(72) Inventor: Louis Causley Causley, San Jose, CA (US)

(21) Appl. No.: 14/150,789

(22) Filed: Jan. 9, 2014

(51) Int. Cl. A61F 7/00

(52) U.S. Cl. CPC A61F 7/00; A61F 2007/006; A61F 2007/0063

(57) ABSTRACT

A device for controlling the environmental area external to post-surgical and non-surgical wounds is disclosed. The device introduces air under pressure to dry and cool the area around the exterior of bandages that are overlaid with skin or are in areas between skin surfaces and that become moist from sweat and accumulate heat. The device can be used with wound coverings ranging from gauze to specialty coverings, and uses available medical air.

### CROSS-REFERENCE TO RELATED APPLICATIONS

This application claims the benefit of U.S. Provisional Applications No. 61/751,256, filed on Jan. 11, 2013, and No. 61/823,387 filed on May 14, 2013. The entire disclosure of these applications is incorporated herein by reference.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### FIELD

This disclosure relates to medical devices, and in particular, to apparatus used to promoting patient comfort by drying and cooling areas associated with surgical and non-surgical wounds.

### BACKGROUND

Despite the availability of many specialized wound covering types, simple gauze bandages are still typical in use as coverings for surgical and non-surgical wounds. The gauze bandage is simply taped over the wound and changed as necessary. Problems occur when these bandages are overlaid by the patient's skin; sweat and heat tend to build up resulting in discomfort to the wearer and increased risk of infection. Examples of skin overlaying the wound area include folds in the areas under arms, under breasts, in the groin area, or when the bandage is between folds of skin. Moisture from the wound plus sweat accumulates in the area, the area can become very warm, and in general conditions can become uncomfortable for the patient. Sometimes powder is applied to the wound area help soak up moisture but it is a temporary measure.

What is needed is a low-cost, effective way to dry the exterior of the wound covering plus dry and cool the area of the skin overlay.

### SUMMARY

A novel aerating device using air under pressure that passes through a perforated plastic membrane that is located between a set of gauze bandages, to dry the wound exterior area, and to cool the area. The device uses sterile air (known as “medical air”) typically available in hospital rooms, and the device is made of low-cost materials and is disposable. The device can be made in various sizes (length and width) to work with standard gauze dressing sizes.

While the prior art includes numerous dressing and wound coverings that have the ability to supply air under pressure, they are all designed to address only the wound itself. In U.S. Pat. No. 7,108,683 Zamierowski discloses a wound covering system that includes the ability to apply vacuum, or air under pressure to the system, as part of a gradient system function. Other prior art such as in U.S. Pat. No. 4,139,004 (Gonzalez) include a cover for the wound itself (replacing a conventional bandage), with a connection for providing air or gases or other substances to the wound itself. These coverings themselves serve as the bandage or dressing and enable air, gases, or liquids to be applied to the wound directly while keeping it covered.

Still other prior art devices provides air bladders with air inlets to compress bandages, such as U.S. Pat. No. 7,935,066 (Shives, et al). Compression is not the function of the present invention.

Brownlee in U.S. Pat. No. 4,898,160 describes a multi-chambered device intended to go inside a surgical cast. This flexible device conforms to the shape of the limb and has an air hose connection, providing air to the device which then distributes it to the skin under the cast. Similar devices exist in prior art and are called “cast coolers”. These inventions typically include structural functions.

The present invention is a different approach. The airflow can be in one or both directions, and uses air to dry excess external moisture from the area external to the wound covering for patient comfort, plus diminish accumulated body heat.

A further understanding of the nature and advantages of the device and methods disclosed herein may be realized by reference to the complete specification and the drawings.

### BRIEF DESCRIPTION OF THE DRAWINGS

FIG. 1A is a top view illustrating the preferred embodiment of the invention.

FIG. 1B is a side view of the invention of FIG. 1A.

FIG. 2 is a drawing showing the flow of air through the device in use.

### DETAILED DESCRIPTION

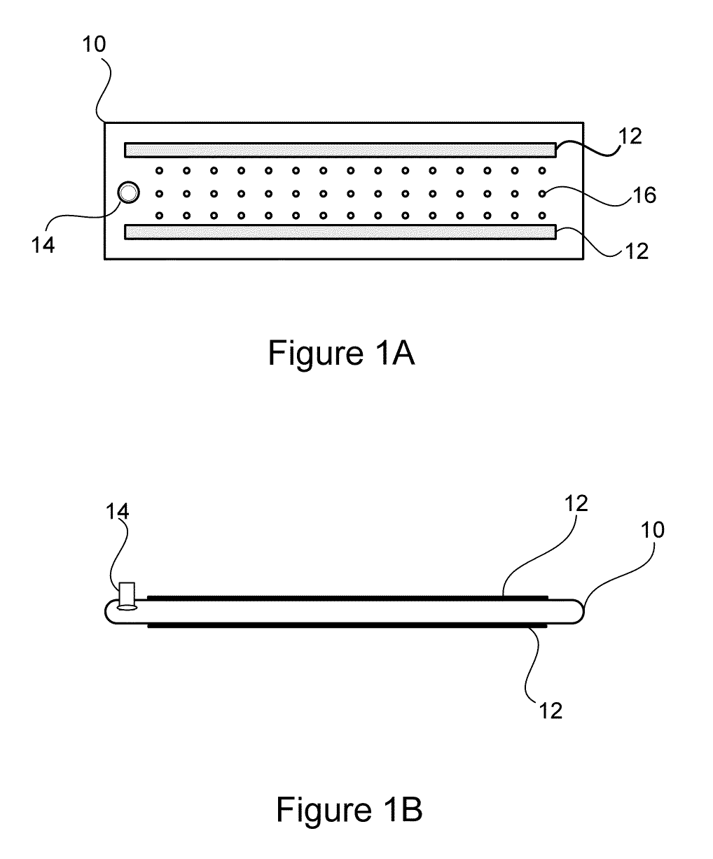

Referring now to FIG. 1A, flexible plastic structure 10 is sealed except for an array of perforations 16 on one or both sides, and connector 14 for the air supply. Structure 10 is a closed pouch of flexible plastic material such as polyethylene or other flexible medical-grade plastic. Perforations 16 are in an array, preferably on both sides of device 10, and are positioned such that air entering through connector 14 exits through perforations 16 evenly and consistently along the length of the array. Structure 10 does not necessarily inflate and expand when air is flowing. Air pressure is adjusted manually at the source to reach the desired level of aeration of structure 10, on a case-by-case basis. Structure 10 can have perforations 16 on one side of structure 10 or both sides of structure 10. Attachment strips 12, a material that adheres to gauze, are included along the length on one or both sides of structure 10. In use, standard sterile gauze is placed onto attachment strips 12, on one or both sides of structure 10, air supply tubing is press-fit onto connector 14, then the assembly is placed over the wound and taped to the patient to hold it in position. Air pressure is adjusted to provide the desired flow rate, determined by medical staff. Should the gauze require changing, structure 10 is removed from the patient, gauze is removed from attachment strips 12, replaced, and then structure 10 is retaped to the patient.

In practice, the length and width of structure 10 can vary from between 3 inches in length and 2 inches in width to as much as 10 inches in length and 5 inches in width, representing a range of sizes to accommodate various gauze sizes.

Referring now to FIG. 1B, a side view is shown of structure 10 with attachment strips 12 on both sides, and air supply connection 14 at one end.

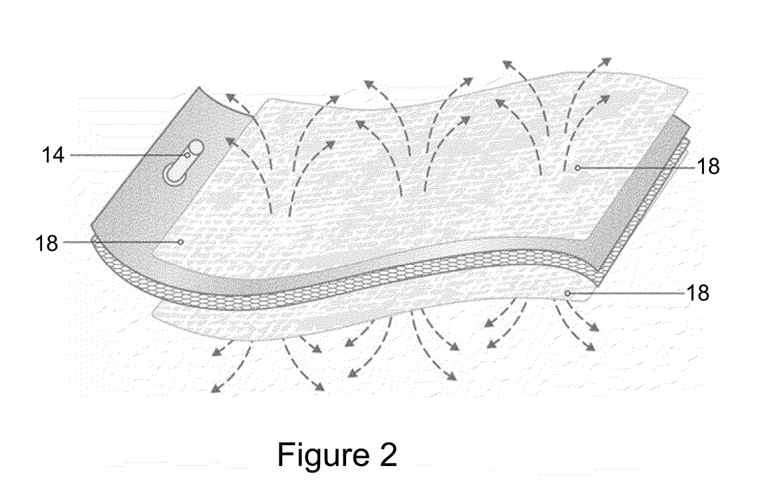

FIG. 2 illustrates the air flow of the preferred embodiment shown in FIG. 1. Air entering connector 14 exits through perforations 16 and through gauze 18. In practice, the gauze 18 placed on the opposite side of the wound will absorb moisture from skin and will be the primary beneficiary of the airflow for drying and cooling. If another type of bandage or wound covering other than gauze is used, the present invention can be placed directly over the wound covering, gauze 12 placed onto the outside, then the entire assembly is taped or otherwise secured to the patient.

The preferred embodiment, and variations accomplished by slight modifications, solves the problems discussed earlier in this document.

In general, those skilled in the art to which this disclosure relates will recognize that many changes in construction and materials as well as differing embodiments will suggest themselves without departing from the spirit and essential characteristics of this disclosure. Accordingly, the disclosures and descriptions herein are intended to be illustrative, and not limiting, of the scope of the invention.

I claim:

1. The device consisting of a perforated flexible plastic enclosure shown in FIGS. 1 and 1A;
a. With a connection for a plastic tube to provide air under pressure.
b. With an array of perforations on one or both sides to enable even distribution of air exiting the perforations.
c. With strips of material for gauze adhesion located along opposing edges of the device, on one or both sides.
